(19) United States
(12) Patent Application Publication
(10) Pub. No.: US 2026/0315801 A1
(43) Pub. Date: Oct. 15, 2026

(54) WIRELESSLY POWERED ELECTRICAL STIMULATION SYSTEMS AND RELATED METHODS

(71) Applicant: MULTI-SCALE MEDICAL ROBOTICS CENTER LIMITED, Hong Kong (CN)

(72) Inventors: Li ZHANG, Hong Kong (CN); Wai Yan Philip CHIU, Hong Kong (CN); Kai Fung CHAN, Hong Kong (CN); Chong ZHANG, Hong Kong (CN); Chengfeng PAN, Hong Kong (CN)

(21) Appl. No.: 18/998,671

(22) PCT Filed: Jul. 26, 2023
(86) PCT No.: PCT/IB2023/057560

§ 371 Date: Jan. 27, 2025

Related U.S. Application Data
(60) Provisional application No. 63/392,506, filed on Jul. 27, 2022.

(51) Int. Cl. A61N 1/36; A61N 1/05; A61N 1/372; A61N 1/375; A61N 1/378

(52) U.S. Cl. CPC A61N 1/36125; A61N 1/0509; A61N 1/0512; A61N 1/36003; A61N 1/36007; A61N 1/36139; A61N 1/3615; A61N 1/36171; A61N 1/36175; A61N 1/37229; A61N 1/37518; A61N 1/3787

(57) ABSTRACT

Systems, devices, and related implantation methods of the wireless-powered electrical stimulator are generally provided. A typical system includes a wearable transmitter for wireless power transfer and an implanted electrical stimulator. Specifically, the electrical stimulator comprises a power management module, a pulse generator, and one or more pairs of electrodes. In some embodiments, the electrical stimulator may include a microcontroller and some sensors detecting related physiological signals of target muscle groups for closed-loop electrical therapy. Moreover, minimally invasive implantation procedures are also disclosed for electrical stimulation therapy through a natural orifice, like the gastrointestinal tract. In some embodiments, electrical stimulation is applied continuously to restore the normal functions of target muscle groups.

### FIELD OF THE INVENTION

The present invention generally relates to wireless-powered medical devices for minimally invasive electrical stimulation therapy to restore the normal functions of typical muscle groups, like the gastrointestinal tract.

### BACKGROUND OF THE INVENTION

Functional electrical stimulation (FES), a method using electrical stimulation to restore the degenerated function of typical muscle groups, has been wildly used in clinical. Various systems, sensors, and algorithms are investigated to improve clinical efficacies. Although transdermal electrical stimulation is a non-invasive method, it needs high voltages or currents to apply effective stimulation across the skin. Meanwhile, implantable stimulators directly apply electrical pulses to control the target muscle group. However, the operation time of the implanted device is limited by the battery capacity, which is directly associated with battery size. Bulk and rigid batteries need invasive implantation with large incisions, while small batteries require frequent replacement, which would increase the risk of infection and other side effects.

Wireless power transfer is a promising solution for implantable medical devices, avoiding the above-mentioned drawbacks. However, the efficiency of wireless power transfer highly depends on the coupling coefficient, which is associated with the orientation, position, and geometry configuration of the receiver coils and transmitter coils. Moreover, efficient power transfer through deep tissue still remains challenging because magnetic field intensity decays quickly as distance increases.

Here, the system, device, and related implantation procedures of the wireless electrical stimulation are provided. In one aspect, the invention includes a wearable power transmitter for wireless power transfer in daily operations. In one aspect, the invention includes an implanted electrical stimulator that is wirelessly powered or wirelessly charged based on magnetic resonance coupling, which prolongs the lifetime and eliminates the need to replace batteries and related potential risks. In some embodiments, the electrical stimulator contains a microcontroller and some sensors for closed-loop electrical stimulation therapies. In one aspect, minimally invasive implantation methods are also disclosed. In some embodiments, endoscopy implantation procedures through natural orifices are provided. It bypasses open surgeries for implantation and thus reduces the risk of infection during recovery.

### SUMMARY OF THE INVENTION

Systems, devices, materials, and related implantation methods are disclosed for the wireless electrical stimulation systems. The system consists of a wearable power transmitter for wireless power transfer and an implanted electrical stimulator with receiver coils. The transmitter coils are placed outside the body and generate alternating magnetic fields for wireless power transfer through deep tissue. The electrical stimulator comprises receiver coils, a power management module, a pulse generator, and one or more pairs of electrodes. In some embodiments, the electrical stimulator further includes sensors detecting related physiological signals of target groups, and a microcontroller for data processing and wireless communications. Moreover, related minimally invasive delivery methods are also provided. The electrical stimulator is delivered through natural orifices by the endoscopy without the need for open surgery or implanted into the abdominal cavity by laparoscopy surgeries with incisions less than 15 mm.

In one aspect, wearable power transmitters are provided. In general, the power transmitter includes a transmitter coil driven by an alternating current or voltage generated by a portable control board. Based on Faraday's law, the transmitter coils generate alternating magnetic fields. Moreover, the transmitter coil is matched with capacitors operating at a resonance frequency ranging from 100 kHz to 1 GH. In some embodiments, the input power and operation frequency of the transmitter coil is controlled by a portable control box. The control box has a rechargeable battery, and a power supply generates a constant current that is converted into an alternating current by a full-bridge inverter. Its switching frequency determines the operation frequency, which is controlled by the square wave signals generated by the control circuits. And the magnetic field intensity shows a linear relationship with the driving currents.

To achieve maximum efficiency, in some embodiments, the transmitter coil is operated at a frequency with peak quality factors, which is primarily associated with the geometric configurations of the transmitter coils. In some embodiments, the transmitter coils are configured with a pair of solenoid shapes, which generates strong and uniform magnetic fields within the region enclosed by coils. In some embodiments, the coils are a pair of Helmholtz coils. In some embodiments, the coils are braided into a planar pad, the magnetic field of which decays quickly as distance increases.

In some embodiments, the transmitter coil is operated at a fixed frequency within industrial, scientific, and medical (ISM) radio bands. A signal generator generates alternating voltage with the operation frequency, and the input power is controlled by a power amplifier. To achieve maximum power transfer efficiency, the impedance of the power transmitter is adjusted to lower return loss due to impedance mismatch. In some embodiments, an antenna tuner consisting of a set of capacitors and inductors is implemented to adjust the transmitter coil's impedance automatically.

In one aspect, the implanted electrical stimulator is provided. It contains receiver coils that compass a closed area. While alternative magnetic fields transmit through the closed loop, an alternating voltage is induced in the receiver coils. Then the alternating voltage is converted to the direct voltage by a full-bridge rectifier. And the voltage with high amplitudes and noises is then regulated to a stable voltage by the power management module. Finally, the pulse generator generates voltage pulses with programmable frequencies, amplitudes, and pulse widths. In some embodiments, a constant current module follows the pulse generator. It converts voltage pulses into current pulses with a constant amplitude while applied to various loads. In some embodiments, the electrical stimulator may also include an onboard antenna operating and a microcontroller unit (MCU) to set these parameters by wireless communications modules.

In some embodiments, the electrical stimulator contains sensors detecting related physiological signals of target muscle groups, including but not limited to electromyography associated with electrical activities of muscle groups, pressure sensors, and strain sensors related to the motility of target muscles.

In some embodiments, a closed-loop stimulation algorithm is implemented. Specifically, the microcontroller regularly measures the physiological signals of target muscle groups by sensors. When abnormal signals are detected, the microcontroller initiates an electrical pulse train with a typical period to restore the normal functions of muscle groups. When the physiological signal returns to normal value, the microcontroller terminates the electrical stimulation.

In some embodiments, the power management module includes a full-bridge rectifier and a linear low dropout regulator (LDO). In some embodiments, the power management module also contains an inverting converter that generates a negative voltage for the pulse generator to synthesize biphasic stimulation signals. In some embodiments, the power management module may also contain a buck converter for more efficient power management.

Fabrication methods of electrodes are generally provided. In some embodiments, the electrodes are fabricated in the form of microneedles that penetrates through the tissues. In some embodiments, the electrodes are planar metal plates made of platinum, gold, iridium, etc. In some embodiments, the surfaces of electrodes are modified by electrodeposition to improve their charge injection capabilities, anti-biofouling capabilities, and biocompatibility.

Fabrication methods of the wireless electrical stimulator and receiving coils are disclosed. To accommodate the motion of target muscles during electrical stimulations, the wireless electrical stimulator is fabricated on flexible substrates such as PDMS, PI, PET, PU and etc. In some embodiments, the circuit trace is fabricated by conductive materials, including but not limited to eutectic gallium and indium (EGaIn), and copper. In some embodiments, the device is encapsulated by a coating material with good biocompatibility and waterproofing, like parylene.

In one aspect, minimally invasive implantation methods and related applications are provided. Natural orifices, like the gastrointestinal tract, urinary tract, etc, are accessible by endoscopies, which provide a minimally invasive method for the implantation or delivery of the electrical stimulator.

In some embodiments, a tunnel is created between the mucosal layer and the muscle layer of the gastrointestinal tract. Then, the wireless electrical stimulator is implanted through the tunnel. After that, the tunnel is sutured or closed by several endo clips.

In some embodiments, the electrical stimulator is integrated into a medical device, like a stent. They are delivered through the natural orifice by a thin catheter. After releasing the stent, the electrical stimulator retains inside the body lumen for a sustained period.

In some embodiments, the electrical stimulator is applied in the gastrointestinal tract to regulate GI disorders, like irritable bowel diseases, gastroesophageal reflux disease, or fecal incontinence.

In some embodiments, the electrical stimulator is used in the bladder or urinary tract to manage urinary incontinence.

In some embodiments, the electrical stimulator is used to restore the normal functions of the pelvic floor muscles.

### BRIEF DESCRIPTION OF THE DRAWINGS

Non-limiting embodiments of the present invention will be described by means of an example combined with figures, which are schematic and are not intended to be drawn to scale. In the figures, each identical or nearly identical component illustrated is typically represented by a single number. For purposes of clarity, not every component is labelled in every figure, nor is every component of each embodiment of the invention shown where illustration is not necessary to allow those of ordinary skill in the art to understand the invention. In the figures:

FIG. 1 shows a typical wireless electrical stimulation system, including a wearable wireless power transmitter and an implanted wireless electrical stimulator. The wireless power transmitter includes a wearable power transmitter and a portable control box. The wireless electrical stimulator is implanted by minimally invasive surgery through a natural orifice, including but not limited to the gastrointestinal tract.

FIG. 2 shows the basic configuration and properties of a power transmitter. FIG. 2a shows a geometric configuration of a typical power transmitter. FIG. 2b shows the 3D magnetic field generated by the power transmitter. FIG. 2c shows the uniform magnetic field distribution within the power transmitter.

FIG. 3 shows a typical design of a portable control board. FIG. 3a shows the block diagram of the portable control box, which includes a rechargeable battery, a power management circuit, a control circuit, and matching capacitors. FIG. 3b shows a typical prototype of the portable control board.

FIG. 4 shows a typical design of the power transmitter. FIG. 4a shows the geometric configuration of the power transmitter. FIG. 4b shows the magnetic field distribution of the power transmitter.

FIG. 5 shows a typical design of the portable control board. FIG. 5a illustrates the block diagram of another portable control box, including a signal generator, a power amplifier, and matching capacitors. FIG. 5b shows a prototype of the proposed portable control board.

FIG. 6 shows a typical block diagram of the implanted wireless electrical stimulator, which includes a receiver coil for wireless charging the onboard battery, a power management module, a pulse stimulation module with one or more pairs of electrodes, and a microcontroller for sensor processing and wireless communication.

FIG. 7 shows biphasic constant current stimulation signals. FIG. 7a shows adjustable constant current stimulations with different load resistances. FIG. 7b shows biphasic constant current pulse stimulations with different load resistances.

FIG. 8a-d show pulse stimulations with programmable frequencies and pulse width.

FIG. 9 shows a typical prototype of the wireless electrical stimulator with a miniature size fabricated by a conventional flexible printed circuit board process.

FIG. 10a-f shows a schematic illustration of the fabrication process of a stretchable circuit using stretchable substrates and liquid metal, like EGaIn.

FIG. 11a-b shows scanning electron microscope (SEM) of electronic components mounted on the stretchable circuits. FIG. 11c-d shows stable interface between components and stretchable circuits against gravity.

FIG. 12 shows images of a stretchable circuit undergoing various deformations, including twist, bend, stretch.

FIG. 13 shows illustrations of two types of receiver coils. FIG. 13a shows illustration of receiver coils with an air core. FIG. 13b shows illustrations of receiver coils with a ferrite core.

FIG. 14a shows an illustration of a air core coil in the form of a stent. FIG. 14b shows an image of an air core coil braided along the skeleton of a stent. FIG. 14c shows an image of a stretched coil up to 50%.

FIG. 15a shows a schematic illustration of a typical wireless electrical stimulator, including a stent, an elastic coil, and a stretchable pulse generator with one or more pairs of electrodes. FIG. 15b shows X-ray images of a non-invasive transoral delivery procedure of wireless electrical stimulation.

FIG. 16 shows a delivery tool that includes a balloon catheter, a block ring, a soft tip, and a flexible shell.

FIG. 17 illustrates the minimally invasive implantation procedure through a natural orifice by an endoscope.

FIG. 18 shows a block diagram for a closed-loop electrical stimulation algorithm.

### DETAILED DESCRIPTION OF THE INVENTION

This invention provides a system for wireless electrical stimulation. In one embodiment, said system comprises: a wearable power transmitter comprising: i) a portable control board; and ii) a transmitter coil for generating alternating magnetic fields with an operation frequency ranging from 100 kHz to 1 GHz for wireless power transfer; and an implanted wireless electrical stimulator comprising: i) one or more pairs of receiver coil for receiving said alternating magnetic fields from the wearable power transmitter and generating an alternating voltage; ii) a power management module for rectifying said alternating voltage to provide stable voltage ranging from −15V to 15V; iii) a pulse generator for generating current pulse signals with programmable amplitudes, frequencies, and pulse widths and iv) one or more pairs of electrodes configured in contact with target muscle groups for delivering said current pulse signals, and v) a microcontroller for data processing and wireless communication.

In one embodiment, the portable control board comprises: i) a rechargeable battery; ii) a power management circuit providing constant currents ranging from 0.1 A to 3 A, and different voltages comprising 5 V and 15V; iii) a control circuit comprising a square wave signal generator with a frequency ranging from 100 kHz to 1 MHz, and metal-oxide-semiconductor field-effect transistor (MOSFET) drivers, a full-bridge inverter consisting of four MOSFETs, and one or more matching capacitors.

In one embodiment, the portable control board comprises: i) a rechargeable battery; ii) a power management circuit; iii) a signal generator for generating alternating signals with a frequency ranging from 1 MHz to 1 GHz; iv) a power amplifier configured with output power ranging from 1 watt to 20 watts; v) an antenna tuner comprising a set of capacitors and inductors that automatically minimizes return loss of the transmitter coil at the operation frequency.

In one embodiment, geometry configurations of the transmitter coils comprises planar, pairs of Helmholtz, single solenoid, or pairs of solenoids with a diameter ranging from 5 cm to 80 cm.

In one embodiment, the implanted wireless electrical stimulator further comprises a wireless charging circuit with charging currents ranging from 5 mA to 100 mA, and a rechargeable battery with a diameter of less than 15 mm, and a thickness of less than 10 mm, and a capacity ranging from 5 mAh to 200 mAh.

In one embodiment, the implanted wireless electrical stimulator further comprises sensors for detecting physiological signals comprising one or more selected from the group consisting of pressure, strain, and electromyography signals of target muscle groups.

In one embodiment, frequency of the current pulse signals range from 1 Hz to 1 K Hz.

In one embodiment, amplitude of the current pulse signals range from 3 mA to 15 mA.

In one embodiment, pulse width of the current pulse signals range from 100 microseconds to 200 milliseconds.

In one embodiment, the receiver coil is fabricated by a single-strand conductive wire; said single-strand conductive wire is made of: i) a material selected from the group consisting of copper, gold, platinum, nitinol alloy; or ii) silicone tubes infilled with liquid metal.

In one embodiment, said liquid metal is eutectic gallium-indium (EGaIn).

In one embodiment, the receiver coil is fabricated in the form of a stent with stretchability ranging from 50% to 200% and a diameter ranging from 16 mm to 28 mm, and a length ranging from 80 mm to 120 mm.

In one embodiment, the receiver coil comprises a ferrite core with relative magnetic permeability ranging from 500 to 300, and is configured with a miniaturized size with a length less than 10 mm, a diameter less than 10 mm.

In one embodiment, the receiver coil is configured with a planar form with a length of less than 100 mm and a width of less than 50 mm.

In one embodiment, the electrodes is fabricated with a needle shape with a length ranging from 100 micrometers to 1 mm and are configured with shapes comprising cones, prims, or pricks.

In one embodiment, the electrodes are fabricated by a conductive material comprising copper, and surface of the electrodes are modified by gold, platinum, and iridium oxide.

In one embodiment, the wireless electrical stimulator is made by: soft materials comprising polydimethylsiloxane (PDMS), styrene ethylene butylene styrene (SEBS), polyurethane (PU) or hydrogels as substrates and encapsulations; and intrinsic conductive materials comprising one or more of silver nanowires, carbon nanotubes, gold nanowires, poly(3,4-ethylenedioxythiophene) polystyrene sulfonate (PEDOT:PSS), or EGaIn.

In one embodiment, This invention also provides an algorithm for closed-loop electrical stimulation therapies implemented in the microcontroller of the wireless electrical stimulator, wherein the microcontroller initiates electrical stimulations when sensors detect abnormal physiological signals, and terminates electrical stimulations after a period of time for at least 20 minutes when the physiological signals return to normal values.

In one embodiment, this invention also provides a minimally invasive method assisted by an endoscopy through natural orifices for implantation of the system of this invention into a tissue with a mucosal layer, a muscle layer and submucosal layer, wherein implantation procedures comprise the following steps: (a) Creating a submucosa tunnel by slicing open mucosal layer with an incision of less than 15 mm; (b) Implanting the wireless electrical stimulator into the submucosa tunnel between the muscle layer and the submucosal layer; (c) Sealing the submucosa tunnel by a set of clips that will detach automatically after a period of time less than 10 days.

In one embodiment, said endoscopy through natural orifices is for gastrointestinal tracts.

In one embodiment, this invention provides a minimally invasive method for implantation of the system of this invention, wherein said receiver coil is fabricated in the form of a stent and delivered through natural orifices by a delivery tool comprising a flexible shell, a flexible tip, a block ring, and a balloon catheter; wherein implantation procedures comprise the following steps: (a) Stretching the wireless electrical stimulator on the stent and inserting into the delivery tool; (b) Inserting the delivery tool carrying the implanted wireless electrical stimulator through the natural orifice; (c) Releasing the electrical stimulator integrated on the stent by pulling the flexible shell of the delivery tool; (d) Inserting electrodes of the wireless electrical stimulator into muscle tissue across a mucosa layer by inflating the balloon catheter of the delivery tool; and (e) Deflating the balloon catheter and retracting the delivery tool through the natural orifice.

In one embodiment, this invention provides a minimally invasive method for implantation of the system of this invention, into an abdominal cavity by laparoscopy surgeries, wherein implantation procedures comprise the following steps: (a) Opening the abdominal cavity with an incision of less than 15 mm; (b) Contacting the electrodes of the wireless electrical stimulator with the target muscle group; (c) Fixing the wireless electrical stimulator by a set of surgical sutures and sealing the abdominal cavity by a series of surgical sutures.

In one embodiment, said target muscle group comprises lower esophagus sphincter, anal sphincter, or fundus of a stomach.

The invention will be better understood by reference to the Experimental Details which follow, but those skilled in the art will readily appreciate that the specific experiments described are only for illustrative purposes and are not meant to limit the invention as described herein, which is defined by the claims that follow thereafter.

Throughout this application, various references or publications are cited. Disclosures of these references or publications in their entireties are hereby incorporated by reference into this application in order to more fully describe the state of the art to which this invention pertains. It is to be noted that the transitional term “comprising”, which is synonymous with “including”, “containing” or “characterized by”, is inclusive or open-ended and does not exclude additional, un-recited elements or method steps.

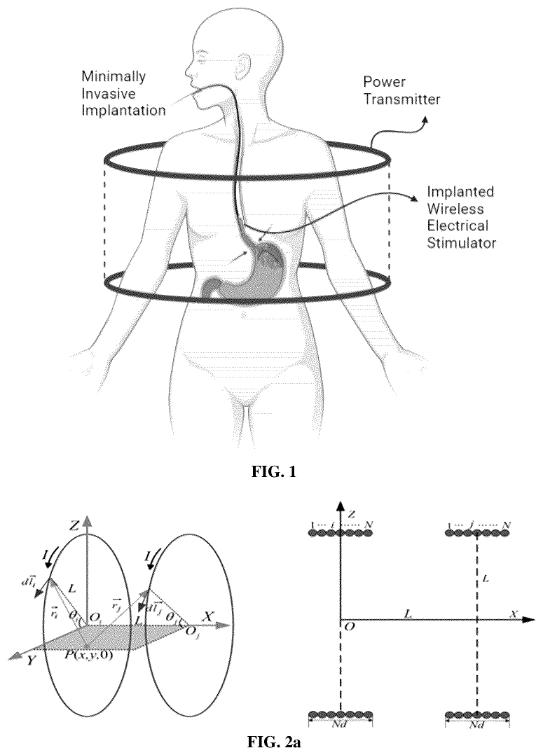

The present invention provides a wireless electrical stimulation system for long-term in vivo electrical stimulation therapy. Referring to FIG. 1, this invention includes two parts, a wearable transmitter with a portable control board for wireless power transfer and a wireless electrical stimulator that applies electrical pulses to restore the normal functions of target muscle groups. Alternating current flows through the transmitter coils that generate alternating magnetic fields. However, the intensity of the magnetic fields generated by a planar coil decay quickly as the distance increases. Moreover, the geometry configuration of the transmitter coil greatly affects the feasibility of in vivo wireless power transfer. Therefore, a transmitter coil with a reasonable configuration is important for wireless power transfer in deep tissues.

### Example 1 A Typical Design of the Wearable Power Transmitter

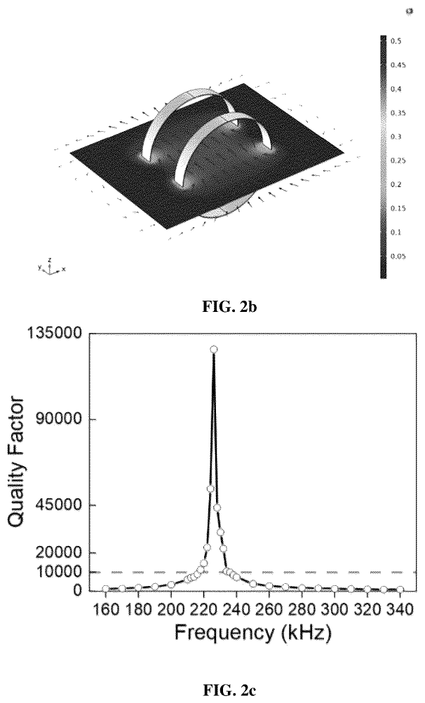

Referring to FIG. 2a, the transmitter coil is a pair of solenoids coil braided by a single conductive wire, including but not limited to copper wires. In some embodiments, the transmitter coil has a diameter ranging from 30 cm to 80 cm, which could accommodate various body shapes. Each side of the solenoids has equal turns, ranging from 5 turns to 40 turns. The interval between two solenoids equals the radius of the power transmitter. Referring to FIG. 2b, this geometric configuration generates a uniform and relatively strong magnetic field within the coil, which is essential to power implants in deep tissue.

For daily operations, the power transmitter coil is fabricated in the form of jackets or wearable overalls. In some embodiments, the power transmitter coil is fabricated on rigid substrates to avoid shape changes, including but not limited to polyvinyl chloride (PVC) tubes and 3D-printed polylactic acid (PLA) shells. In some embodiments, the transmitter coil is configured in an ellipsoid shape to accommodate various body shapes.

The input power of the transmitter coil is primarily limited by the specific absorption rate (SAR), a safety evaluation criterion for wireless power transfer. To minimize power loss due to the transmitter coil's internal impedance, the power transmitter is operated near the frequency with a peak quality factor, which is associated with the geometric configuration of the power transmitter. Referring to FIG. 2c, the peak quality factor is found by sweeping frequency in operational ranges.

### Example 2 A Typical Design of the Portable Control Board

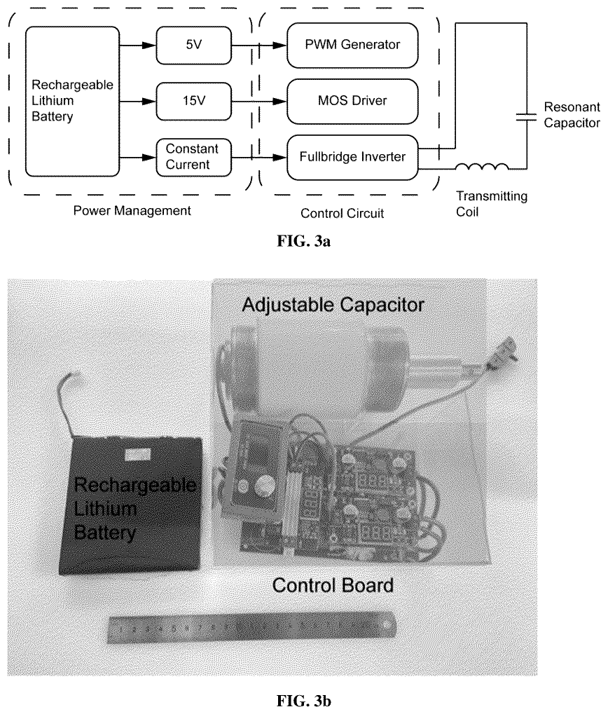

Referring to FIG. 3a, the portable control board includes a rechargeable battery, a power management module, a control circuit, and adjustable matching capacitors. The control circuit includes a pulse width modulation (PWM) generator providing square wave signals with adjustable frequencies ranging from 100 kHz to 1 M Hz and controls switching frequencies of a full-bridge inverter, which converts direct currents into alternative currents and then drives the transmitter coil. To achieve high power transfer efficiency, the transmitter coil is matched to the operating frequency with the peak quality factor by adjusting matching capacitors. Moreover, magnetic fields show a linear relationship with driving currents. The input power is adjusted by controlling the constant current flew into the full-bridge inverter. Referring to FIG. 3b, a typical prototype of the control board is disclosed. It includes a rechargeable battery that can power the system for a sustained period from 8 hours to over 24 hours, depending on the driving currents. In some embodiments, the large adjustable capacitors can be replaced by a series of onboard capacitors to minimize the overall size of the control board.

### Example 3 An Alternative Design of the Power Transmitter

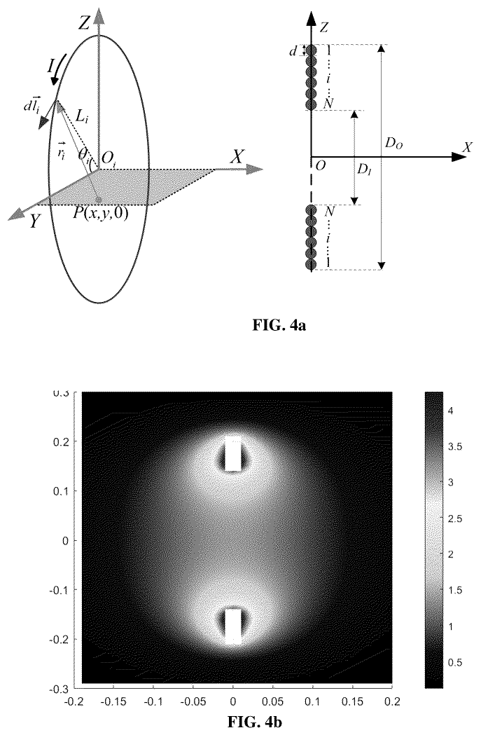

In some embodiments, the transmitter coil has a planar form matched with a resonant frequency ranging from 1 MHz to 100 MHz. Referring to FIG. 4a, it is configured in the form of a coplanar concentric coil braided by a single conductive wire. Referring to FIG. 4b, the magnetic field generated by this planar coil decreases as distance increases from the centre point. In some embodiments, the transmitter coil has an outer diameter ranging from 10 cm to 50 cm. In some embodiments, the transmitter coil is placed parallel to the chest of a subject. In some embodiments, a subject is located at the centre of the transmitter coil.

Despite various geometry configurations, this planar coil is matched to a typical resonant frequency such as 13.56 MHz. A corresponding driving circuit is disclosed. Referring to FIG. 5a, the driving circuit includes a rechargeable battery, a signal generator that provides alternating voltage signals at the resonant frequency, and a power amplifier that amplifies the input voltage to the transmitter coil. Referring to FIG. 5b, a typical prototype is disclosed. This circuit operates at a fixed frequency and fixed input power but offers a lightweight and miniature form factor. Moreover, the transmitter coil is sensitive to the environment because the resonant frequency would drift due to parasitic capacitance. In some embodiments, an antenna tuner consists of a reflection bridge and a set of capacitors and inductors. It automatically adjusts the impedance of the transmitter coil to minimize power loss due to resonant frequency drift.

### Example 4 General Design of the Implanted Wireless Electrical Stimulator

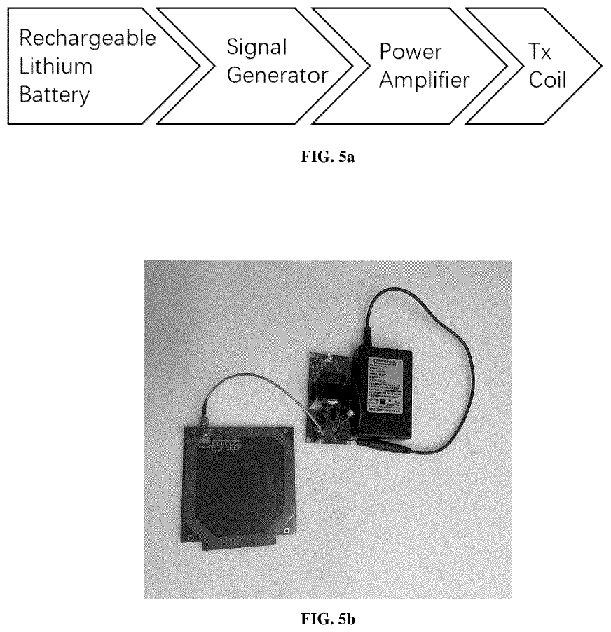

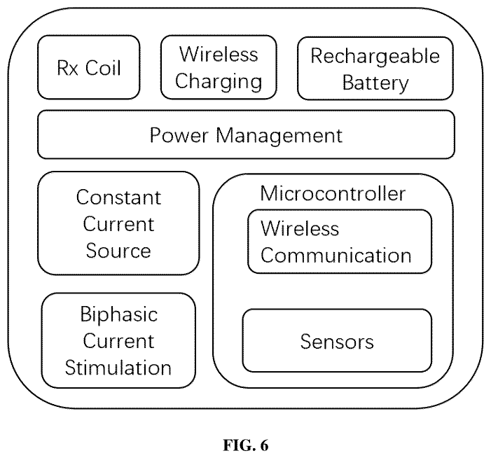

Referring to FIG. 6, the wireless electrical stimulator generally contains three parts, a power management module, a stimulation module, and may further include a sensing module. In some embodiments, the wireless electrical stimulator is fully wirelessly powered, which needs continuous operation of the wearable power transmitter. The power management module includes power management integrated circuits (ICs), and a receiver coil. The receiver coil induces alternative voltage within the time-variant magnetic fields. In some embodiments, the alternative voltage is converted into direct voltage by a rectifier circuit such as but not limited to a full bridge converter. The converted voltage is smoothed by a capacitor. Furthermore, a linear low dropout regulator (LDO) regulates the voltage to a stable voltage. In some embodiments, an inverting converter generates negative voltages for the stimulation modules to generate biphasic stimulations. In some embodiments, the wireless electrical stimulator is battery-powered and wirelessly charged. The power management module further includes a rechargeable battery and related charging circuits. In some embodiments, the wireless electrical stimulator contains a rechargeable battery that can be wirelessly recharged. It has a capacity ranging from 1 mAh to 2000 mAh to power the whole circuit for a typical period, ranging from 5 minutes to 24 hours. Meanwhile, battery capacity is directly associated with battery size. In some embodiments, the rechargeable battery has a thickness of less than 6 mm and a diameter of less than 12 mm. In some embodiments, the wireless charging circuit has a charging current ranging from 5 mA to 400 mA.

In some embodiments, the sensing module contains electrodes measuring the electromyography of target muscle groups. In some embodiments, the sensing module contains pressure sensors measuring internal lumen pressure. In some embodiments, the sensing module includes a strain sensor measuring muscle motion directly. In some embodiments, the stimulator also contains a wireless communication module that includes a microcontroller and an antenna for data processing and wireless communication.

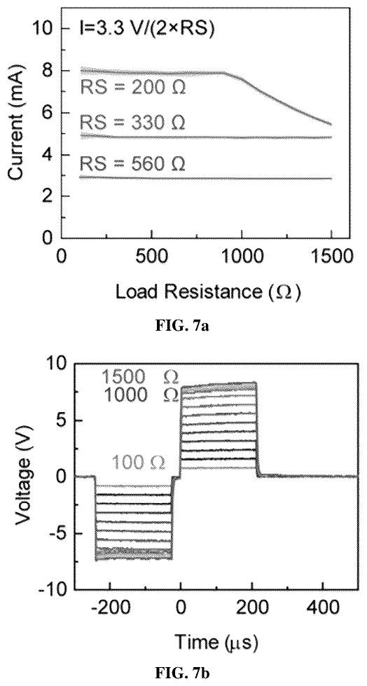

In general, a pulse train consists of a series of pulse signals with constant intervals and pulse width. In some embodiments, the stimulation signals are voltage pulses. It applies pulses with constant voltage amplitudes to the tissue. Due to the parasitic capacitance between tissue-electrode, the charging current varies during stimulation. Overcharging has related safety issues. Referring to FIG. 7, in some embodiments, the stimulation module contains a constant current module that converts voltage pulses into constant current pulses with different loads, ranging from 3 mA to 10 mA. In some embodiments, the pulses are monophasic signals. However, the accumulated charge may lead to muscle fatigue and electrode damage. To achieve net zero currents, charge-balanced stimulation is implemented by biphasic current pulses referring to FIG. 7b.

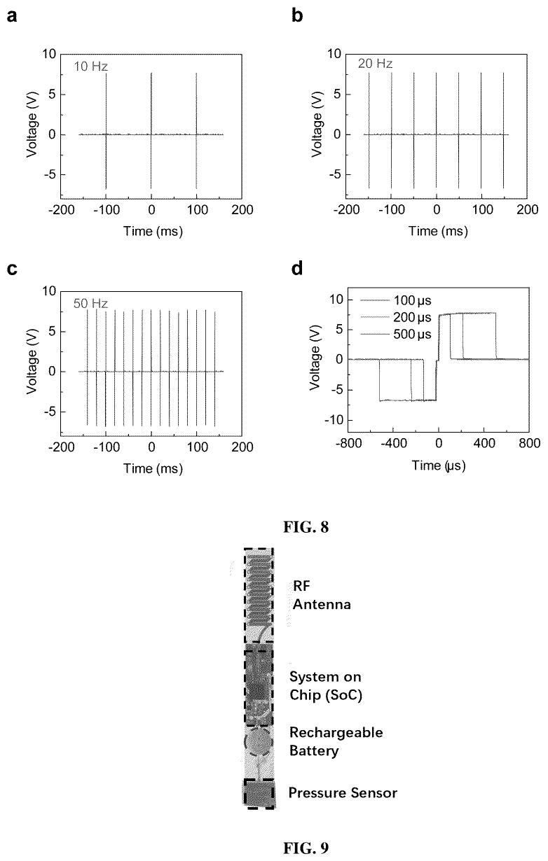

According to previous research, amplitudes, frequency, and pulse width, all affect the efficacy of the electrical stimulation therapy. Referring to FIG. 8, the frequency is controlled by the voltage signals generated by the microcontroller, ranging from 1 Hz to 1000 Hz. The pulse width is programmable from 50 microseconds to 500 milliseconds. Combined with the wireless communication module, all stimulation parameters can be programmed wirelessly.

### Example 5 Fabrication Methods of the Wireless Electrical Stimulator

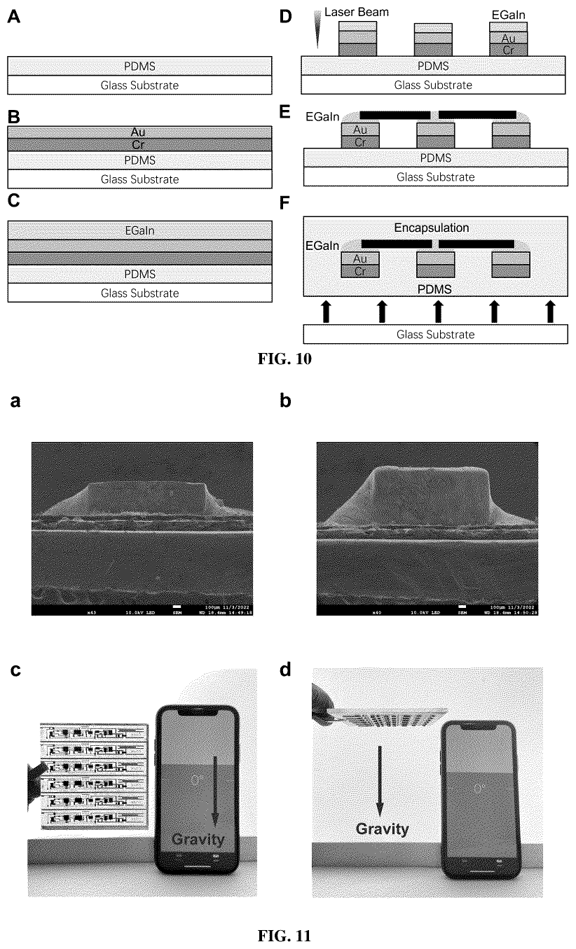

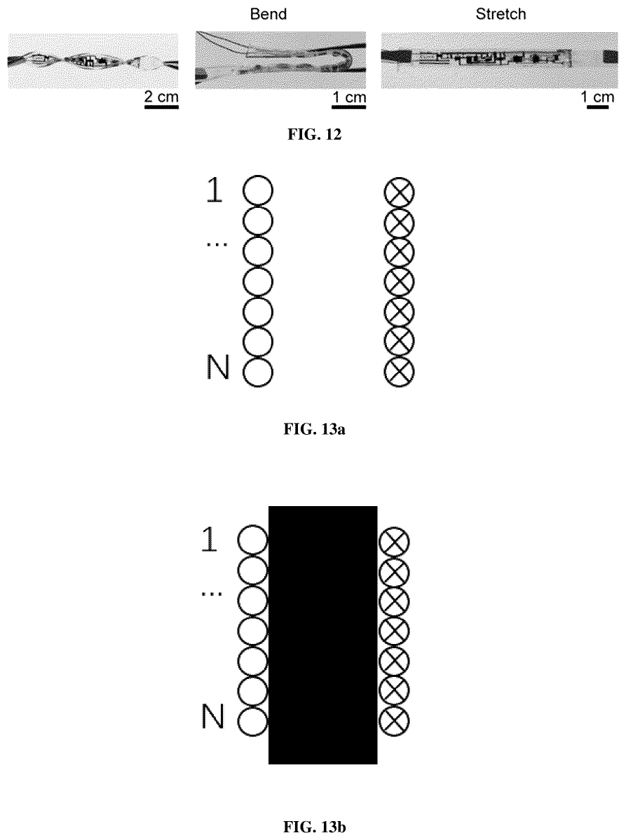

In one aspect, the fabrication methods of the wireless electrical stimulator are disclosed. To accommodate muscle movements during stimulations, the wireless electrical stimulator is fabricated in a flexible form factor. In some embodiments, the circuit is fabricated by conventional flexible PCB schemes on flexible substrates like polyimide referring to FIG. 9. In some embodiments, the circuit is further fabricated on a stretchable substrate, including but not limited to polydimethylsiloxane (PDMS), polyurethane (PU), and styrene ethylene butylene styrene (SEBS), referring to FIG. 10. The insulator layer has a thickness ranging from 10 micrometers to 500 micrometers. After that, a conductive layer is deposited on top of the insulator layer. Materials of the conductive layer include but are not limited to copper, gold, titanium, platinum, and chromium. The thickness of the conductive layer ranges from 50 nm to 200 nm. In some embodiments, EGaIn solution with 3 wt % NaOH solution is poured over the conductive layer to form an alloy with intrinsic stretchability. To form circuit traces, a laser is used to etch away redundant parts. In some embodiments, the wavelength of the laser ranges from 355 nm to 1064 nm. The linewidth fabricated is at least greater than 10 micrometers. After cleaning and drying, electronic components are placed gently over the EGaIn trace. Finally, the whole circuit is encapsulated with an insulating material, like PDMS and parylene. Referring to FIG. 11, the large surface tension of EGaIn provides additional force and ensures stable electrical contact between components and circuit traces. Referring to FIG. 12, the fabricated circuit can undergo various deformations, including bending, twisting, and stretching, which is suitable for applications with muscle motions.

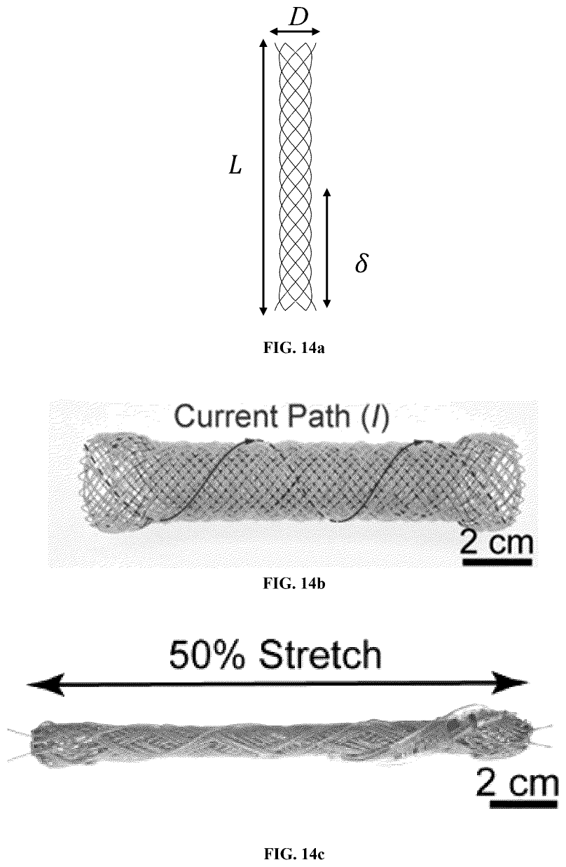

In one aspect, the fabrication methods of receiver coils are disclosed. In general, the receiver coil has two types, including air core coils referring to FIG. 13a and ferrite core coils referring to FIG. 13b. In some embodiments, the air core coil is fabricated in the form of a planar coil on a flexible or stretchable substrate, as mentioned before. In some embodiments, the air core coil is braided by a single-strand conductive coil in the form of a stent referring to FIG. 14a. The conductive materials include but are not limited to nitinol alloy covered with an insulating layer. In some embodiments, the air core coil is braided by a single-strand conductive coil along the skeleton of a stent referring to FIG. 14b. The conductive materials include but are not limited to silicone tubes infilled with EGaIn. Referring to FIG. 14c, the excellent mechanical properties and parallel mesh jointly enable good stretchability of the air core coil compliant with various deformations inside the body. In some embodiments, the receiver coil contains a ferrite core with high relative magnetic permeability. It offers a smaller size to achieve the same voltage. In some embodiments, the receiving coils have a diameter ranging from 4 mm to 10 mm and a length ranging from 2 mm to 10 mm. In some embodiments, the receiving coils are matched to a resonant frequency ranging from 1 MHz to 100 MHz.

### Example 6 Non-Invasive Transoral Delivery of the Wireless Electrical Stimulator

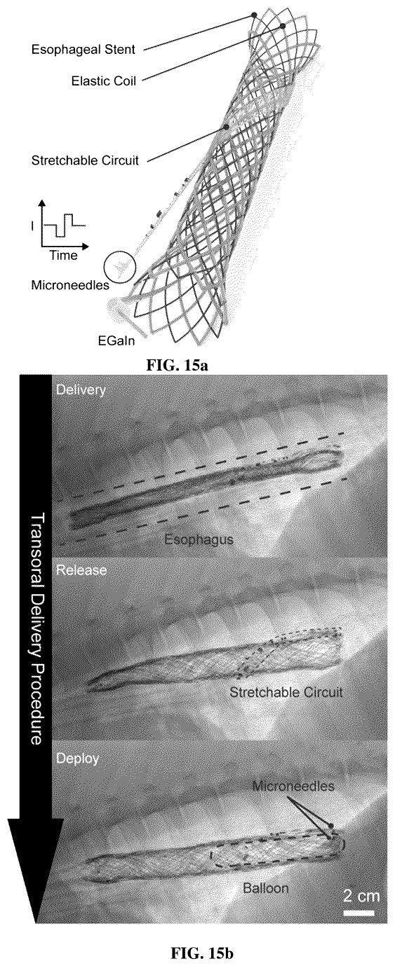

In one aspect, non-invasive endoscopy delivery through the natural orifice is provided. In some embodiments, the wireless electrical stimulator is integrated on a stent referring to FIG. 15a. It shows good mechanical properties with stretchability up to 50%. Referring to FIG. 15b, the stent with the electrical stimulator is compressed within the delivery catheter. Then, the catheter is delivered through the natural orifice. After releasing the stent, the self-expandable stent structure helps the device retain inside the narrow channel. Finally, a balloon catheter is inflated to fully release the wireless electrical stimulator and provide electrodes tightly contact with tissues. In some embodiments, the electrodes are made in the form of microneedles, which are then inserted across the mucosal layer and the needle tips contact with the muscle layer. The length of the microneedle ranges from 300 μm to 800 μm. The diameter of the microneedle ranges from 100 μm to 600 μm. In some embodiments, the electrodes are mounted on the out layer of the stent. They direct contact with the tissue since the stent applies normal force to the tissue.

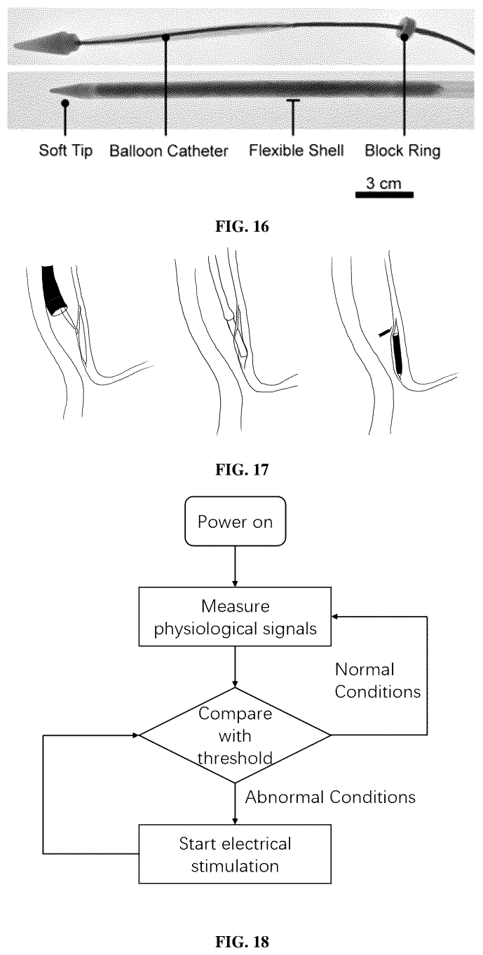

Referring to FIG. 16, the delivery catheter for the non-invasive endoscopy delivery is provided. The delivery catheter generally consists of a balloon catheter, a soft tip, a block ring, and a flexible shell. The stent is compressed and loaded between the flexible tube and the cover tube. During the operation, the surgeon holds the distal end of the flexible tube and pulls the cover tube to release the stent. The block ring prevents the retraction of the stent due to friction during the release process. In some embodiments, the procedure is conducted under X-ray. The radio marker shows the position of the stent under the X-ray image. In some embodiments, the delivery catheter includes a small camera located at the head of the flexible tip. With the illuminance of LED arrays around the camera, it provides an endoscopic view for the operation, which avoids exposure to ionizing radiation and also increases the success rate of the delivery procedure in the dark in vivo environment.

### Example 6 Minimally Invasive Implantation Method of the Wireless Rechargeable Electrical Stimulator

In one aspect, the endoscopy procedure for minimally invasive implantation is also disclosed. In some embodiments, the wireless electrical stimulator is implanted through a natural orifice, including but not limited to the gastrointestinal (GI) tract. Referring to FIG. 17, the endoscopy is inserted through the esophagus. An initial incision will be made to create a tunnel between the mucosa and muscle layers. Then, the electrical stimulator will be delivered and implanted inside the tunnel. The exposed electrodes direct contact with the esophageal muscle for effective electrical stimulation. At the end of the procedure, the incision will be closed with endoscopic clips. After a period of time over 5 days, the endoscopic clips will detach automatically due to tissue regeneration.

In some embodiments, the wireless electrical stimulator is implanted inside the abdominal cavity by laparoscopy. The abdominal cavity is opened with an incision of less than 15 mm. And electrodes of the wireless electrical stimulator are configured in contact with the target muscle group, including but not limited to the lower esophagus sphincter, anal sphincter, and fundus of the stomach. Finally, the wireless electrical stimulator is fixed by a set of surgical sutures and the abdominal cavity is sealed by a series of surgical sutures.

### Example 7 An Algorithm of Closed-Loop Electrical Stimulation Therapy

Referring to FIG. 18, a closed-loop electrical stimulation algorithm is implemented into the microcontroller. When abnormal signals are detected, the microcontroller initiates electrical stimulation pulses for a period of time to restore the normal functions of target muscle groups. The electrical stimulation session stops after the physiological signal returns to normal value. In some embodiments, the detected signal is sent out by wireless communication modules for further analysis. In some embodiments, the embedded sensor and this algorithm automatically control the electrical stimulation without interventions.

In some embodiments, the electrical stimulator is implanted in the lower esophagus sphincter for the treatment of GERD. In some embodiments, the electrical stimulator is implanted near the anal sphincter for the treatment of fecal incontinence. In some embodiments, the electrical stimulator is delivered through the urinary tract and implanted in the bladder for the treatment of urinary incontinence. The physiological signal refers to pressure at the target site measured by the pressure sensor. When the pressure drops below a threshold, the microcontroller initiates a session of electrical stimulations to increase the pressure back to normal values.

In some embodiments, the electrical stimulator is implanted in the stomach for the treatment of gastroparesis. The strain sensor is placed and fixed on the fundus of the stomach. When the stomach is full, the fundus is stretched due to volume change. After detecting a strain change over a threshold, the wireless electrical stimulator starts a period of electrical stimulation. It invokes stomach movement to help food digestion. The microcontroller stops the stimulation when the strain value returns to normal value, indicating the stomach is empty.

Related applications for the wireless electrical stimulation system and minimally invasive implantation procedures are also provided. The natural orifice includes but is not limited to the gastrointestinal tract, urinary tract, trachea, vagina, etc. In some embodiments, the electrical stimulator is implanted in the small intestine for the treatment of irritable bowel disease. In some embodiments, the electrical stimulator is implanted to restore the normal functions of the pelvic floor muscles. In some embodiments, the physiological signal refers to electromyography measured by one or more pairs of electrodes.

What is claimed is:

1. A system for wireless electrical stimulation, comprising:
a wearable power transmitter comprising:
i. a portable control board; and
ii. a transmitter coil for generating alternating magnetic fields with an operation frequency ranging from 100 kHz to 1 GHz for wireless power transfer; and
an implanted wireless electrical stimulator comprising:
i. one or more pairs of receiver coil for receiving said alternating magnetic fields from the wearable power transmitter and generating an alternating voltage;
ii. a power management module for rectifying said alternating voltage to provide stable voltage ranging from −15V to 15V;
iii. a pulse generator for generating current pulse signals with programmable amplitudes, frequencies, and pulse widths and
iv. one or more pairs of electrodes configured in contact with target muscle groups for delivering said current pulse signals, and
v. a microcontroller for data processing and wireless communication.

2. The system of claim 1, wherein the portable control board comprises:
i. a rechargeable battery;
ii. a power management circuit providing constant currents ranging from 0.1 A to 3 A, and different voltages comprising 5 V and 15V;
iii. a control circuit comprising a square wave signal generator with a frequency ranging from 100 kHz to 1 MHz, and metal-oxide-semiconductor field-effect transistor (MOSFET) drivers, a full-bridge inverter consisting of four MOSFETs, and one or more matching capacitors.

3. The system of claim 1, wherein the portable control board comprises:
i. a rechargeable battery;
ii. a power management circuit;
iii. a signal generator for generating alternating signals with a frequency ranging from 1 MHz to 1 GHz;
iv. a power amplifier configured with output power ranging from 1 watt to 20 watts;
v. an antenna tuner comprising a set of capacitors and inductors that automatically minimizes return loss of the transmitter coil at the operation frequency.

4. The system of claim 1, wherein geometry configurations of the transmitter coils comprises planar, pairs of Helmholtz, single solenoid, or pairs of solenoids with a diameter ranging from 5 cm to 80 cm.

5. The system of claim 1, wherein the implanted wireless electrical stimulator further comprises a wireless charging circuit with charging currents ranging from 5 mA to 100 mA, and a rechargeable battery with a diameter of less than 15 mm, and a thickness of less than 10 mm, and a capacity ranging from 5 mAh to 200 mAh.

6. The system of claim 1, wherein the implanted wireless electrical stimulator further comprises sensors for detecting physiological signals comprising one or more selected from the group consisting of pressure, strain, and electromyography signals of target muscle groups.

7. The system of claim 1, wherein frequency of the current pulse signals range from 1 Hz to 1 K Hz.

8. The system of claim 1, wherein amplitude of the current pulse signals range from 3 mA to 15 mA.

9. The system of claim 1, wherein pulse width of the current pulse signals range from 100 microseconds to 200 milliseconds.

10. The system of claim 1, wherein the receiver coil is fabricated by a single-strand conductive wire; said single-strand conductive wire is made of:
i. a material selected from the group consisting of copper, gold, platinum, nitinol alloy; or
ii. silicone tubes infilled with liquid metal.

11. The system of claim 10, wherein said liquid metal is eutectic gallium-indium (EGaIn).

12. The system of claim 1, wherein the receiver coil is fabricated in the form of a stent with stretchability ranging from 50% to 200% and a diameter ranging from 16 mm to 28 mm, and a length ranging from 80 mm to 120 mm.

13. The system of claim 1, wherein the receiver coil comprises a ferrite core with relative magnetic permeability ranging from 500 to 300, and is configured with a miniaturized size with a length less than 10 mm, a diameter less than 10 mm.

14. The system of claim 1, wherein the receiver coil is configured with a planar form with a length of less than 100 mm and a width of less than 50 mm.

15. The system of claim 1, wherein the electrodes is fabricated with a needle shape with a length ranging from 100 micrometers to 1 mm and are configured with shapes comprising cones, prims, or pricks.

16. The system of claim 1, wherein the electrodes are fabricated by a conductive material comprising copper, and surface of the electrodes are modified by gold, platinum, and iridium oxide.

17. The system of claim 1, wherein the wireless electrical stimulator is made by:
soft materials comprising polydimethylsiloxane (PDMS), styrene ethylene butylene styrene (SEBS), polyurethane (PU) or hydrogels as substrates and encapsulations; and
intrinsic conductive materials comprising one or more of silver nanowires, carbon nanotubes, gold nanowires, poly(3,4-ethylenedioxythiophene) polystyrene sulfonate (PEDOT:PSS), or EGaIn.

18. An algorithm for closed-loop electrical stimulation therapies implemented in the microcontroller of the wireless electrical stimulator, wherein the microcontroller initiates electrical stimulations when sensors detect abnormal physiological signals, and terminates electrical stimulations after a period of time for at least 20 minutes when the physiological signals return to normal values.

19. A minimally invasive method assisted by an endoscopy through natural orifices for implantation of the system of claim 1 into a tissue with a mucosal layer, a muscle layer and submucosal layer, wherein implantation procedures comprise the following steps:
(a) Creating a submucosa tunnel by slicing open mucosal layer with an incision of less than 15 mm.
(b) Implanting the wireless electrical stimulator into the submucosa tunnel between the muscle layer and the submucosal layer.
(c) Sealing the submucosa tunnel by a set of clips that will detach automatically after a period of time less than 10 days.

20. The method of claim 19, wherein said endoscopy through natural orifices is for gastrointestinal tracts.

21. A minimally invasive method for implantation of the system of claim 1, wherein said receiver coil is fabricated in the form of a stent and delivered through natural orifices by a delivery tool comprising a flexible shell, a flexible tip, a block ring, and a balloon catheter; wherein implantation procedures comprise the following steps:
(a) Stretching the wireless electrical stimulator on the stent and inserting into the delivery tool;
(b) Inserting the delivery tool carrying the implanted wireless electrical stimulator through the natural orifice;
(c) Releasing the electrical stimulator integrated on the stent by pulling the flexible shell of the delivery tool;
(d) Inserting electrodes of the wireless electrical stimulator into muscle tissue across a mucosa layer by inflating the balloon catheter of the delivery tool; and
(e) Deflating the balloon catheter and retracting the delivery tool through the natural orifice.

22. A minimally invasive method for implantation of the system of claim 1, into an abdominal cavity by laparoscopy surgeries, wherein implantation procedures comprise the following steps:
(a) Opening the abdominal cavity with an incision of less than 15 mm;
(b) Contacting the electrodes of the wireless electrical stimulator with the target muscle group;
(c) Fixing the wireless electrical stimulator by a set of surgical sutures and sealing the abdominal cavity by a series of surgical sutures.

23. The method of claim 22, wherein said target muscle group comprises lower esophagus sphincter, anal sphincter, or fundus of a stomach.
